SEQUENCE LISTING (45 sequences)

SEQ ID NO: 1 - DNA, 25 bp, synthetic construct
  source 1..25: mol_type = other DNA
gcttcggcagcacatatactaaaat

SEQ ID NO: 2 - DNA, 23 bp, synthetic construct
  source 1..23: mol_type = other DNA
cgcttcacgaatttgcgtgtcat

SEQ ID NO: 3 - DNA, 21 bp, synthetic construct
  source 1..21: mol_type = other DNA
cgggctctttggttatctagc

SEQ ID NO: 4 - DNA, 20 bp, synthetic construct
  source 1..20: mol_type = other DNA
cagccacaaaagagcacaat

SEQ ID NO: 5 - RNA, 23 nt, synthetic construct
  source 1..23: mol_type = other RNA
tctttggttatctagctgtatga

SEQ ID NO: 6 - RNA, 23 nt, synthetic construct
  source 1..23: mol_type = other RNA
atacagctagataaccaaagatt

SEQ ID NO: 7 - DNA, 22 bp, synthetic construct
  source 1..22: mol_type = other DNA
gccgagtctttggttatctagc

SEQ ID NO: 8 - DNA, 20 bp, synthetic construct
  source 1..20: mol_type = other DNA
agtgcagggtccgaggtatt

SEQ ID NO: 9 - DNA, 22 bp, synthetic construct
  source 1..22: mol_type = other DNA
gccaccttttgacagtgatgag

SEQ ID NO: 10 - DNA, 20 bp, synthetic construct
  source 1..20: mol_type = other DNA
aaggtccacgggaaagacac

SEQ ID NO: 11 - DNA, 22 bp, synthetic construct
  source 1..22: mol_type = other DNA
gtccttcctaccccaatttcca

SEQ ID NO: 12 - DNA, 20 bp, synthetic construct
  source 1..20: mol_type = other DNA
taacgcactaggtttgccga

SEQ ID NO: 13 - DNA, 25 bp, synthetic construct
  source 1..25: mol_type = other DNA
catcttctcaaaattcgagtgacaa

SEQ ID NO: 14 - DNA, 23 bp, synthetic construct
  source 1..23: mol_type = other DNA
tgggagtagacaaggtacaaccc

SEQ ID NO: 15 - DNA, 20 bp, synthetic construct
  source 1..20: mol_type = other DNA
agagggaaatcgtgcgtgac

SEQ ID NO: 16 - DNA, 21 bp, synthetic construct
  source 1..21: mol_type = other DNA
caatagtgatgacctggccgt

SEQ ID NO: 17 - DNA, 23 bp, synthetic construct
  source 1..23: mol_type = other DNA
cgtgaagaagaaggcccgcaagt

SEQ ID NO: 18 - DNA, 25 bp, synthetic construct
  source 1..25: mol_type = other DNA
cttggaggcggcaacagctttagta

SEQ ID NO: 19 - DNA, 20 bp, synthetic construct
  source 1..20: mol_type = other DNA
cgggctgagaactgaattcc

SEQ ID NO: 20 - DNA, 20 bp, synthetic construct
  source 1..20: mol_type = other DNA
cagccacaaaagagcacaat

SEQ ID NO: 21 - DNA, 20 bp, synthetic construct
  source 1..20: mol_type = other DNA
cgggctccctgagaccctaa

SEQ ID NO: 22 - DNA, 20 bp, synthetic construct
  source 1..20: mol_type = other DNA
cagccacaaaagagcacaat

SEQ ID NO: 23 - DNA, 20 bp, synthetic construct
  source 1..20: mol_type = other DNA
cgggctggcagtgtcttagc

SEQ ID NO: 24 - DNA, 20 bp, synthetic construct
  source 1..20: mol_type = other DNA
cagccacaaaagagcacaat

SEQ ID NO: 25 - DNA, 20 bp, synthetic construct
  source 1..20: mol_type = other DNA
cgggcaggcggagacttggg

SEQ ID NO: 26 - DNA, 20 bp, synthetic construct
  source 1..20: mol_type = other DNA
cagccacaaaagagcacaat

SEQ ID NO: 27 - DNA, 19 bp, synthetic construct
  source 1..19: mol_type = other DNA
cgggccggggccgtagcac

SEQ ID NO: 28 - DNA, 20 bp, synthetic construct
  source 1..20: mol_type = other DNA
cagccacaaaagagcacaat

SEQ ID NO: 29 - DNA, 21 bp, synthetic construct
  source 1..21: mol_type = other DNA
cgggcttaatgctaattgtga

SEQ ID NO: 30 - DNA, 20 bp, synthetic construct
  source 1..20: mol_type = other DNA
cagccacaaaagagcacaat

SEQ ID NO: 31 - DNA, 20 bp, synthetic construct
  source 1..20: mol_type = other DNA
cgggctagcagcacatcatg

SEQ ID NO: 32 - DNA, 20 bp, synthetic construct
  source 1..20: mol_type = other DNA
cagccacaaaagagcacaat

SEQ ID NO: 33 - DNA, 20 bp, synthetic construct
  source 1..20: mol_type = other DNA
cgggctggcagtgtcttagc

SEQ ID NO: 34 - DNA, 20 bp, synthetic construct
  source 1..20: mol_type = other DNA
cagccacaaaagagcacaat

SEQ ID NO: 35 - DNA, 20 bp, synthetic construct
  source 1..20: mol_type = other DNA
cgggctagcagcacatcatg

SEQ ID NO: 36 - DNA, 20 bp, synthetic construct
  source 1..20: mol_type = other DNA
cagccacaaaagagcacaat

SEQ ID NO: 37 - DNA, 21 bp, synthetic construct
  source 1..21: mol_type = other DNA
cgggctaaagtgcttatagtg

SEQ ID NO: 38 - DNA, 20 bp, synthetic construct
  source 1..20: mol_type = other DNA
cagccacaaaagagcacaat

SEQ ID NO: 39 - DNA, 21 bp, synthetic construct
  source 1..21: mol_type = other DNA
cgggccaaagtgctaacagtg

SEQ ID NO: 40 - DNA, 20 bp, synthetic construct
  source 1..20: mol_type = other DNA
cagccacaaaagagcacaat

SEQ ID NO: 41 - RNA, 21 nt, synthetic construct
  source 1..21: mol_type = other RNA
gcagtttgatgctgatgaatt

SEQ ID NO: 42 - RNA, 21 nt, synthetic construct
  source 1..21: mol_type = other RNA
ttcatcagcatcaaactgctt

SEQ ID NO: 43 - RNA, 21 nt, synthetic construct
  source 1..21: mol_type = other RNA
gattgctgaagaagttcaatt

SEQ ID NO: 44 - RNA, 21 nt, synthetic construct
  source 1..21: mol_type = other RNA
ttgaacttcttcagcaatctt

SEQ ID NO: 45 - DNA, 49 bp, synthetic construct
  source 1..49: mol_type = other DNA
cctgttgtctccagccacaaaagagcacaatatttcaggagacaacagg

(19) United States
(12) Patent Application Publication
(10) Pub. No.: US 2024/0425865 A1
(43) Pub. Date: Dec. 26, 2024

(54) METHODS FOR TREATING CELLULAR INFLAMMATION INDUCED BY AFB1

(71) Applicant: HEFEI UNIVERSITY OF TECHNOLOGY, Hefei (CN)

(72) Inventors: Wenda WU, Hefei (CN); Jie ZHANG, Hefei (CN); Tushuai LI, Hefei (CN); Baocai XU, Hefei (CN); Zihui QIN, Hefei (CN); Huiping XIAO, Hefei (CN); Xiaojie MENG, Hefei (CN)

(73) Assignee: HEFEI UNIVERSITY OF TECHNOLOGY, Hefei (CN)

(21) Appl. No.: 18/749,562

(22) Filed: Jun. 20, 2024

(30) Foreign Application Priority Data
Jun. 20, 2023 (CN) 202310730348.5

(51) Int. Cl. C12N 15/113; C12Q 1/686; C12Q 1/6883; G01N 33/68

(52) U.S. Cl. CPC C12N 15/1137; C12Q 1/686; C12Q 1/6883; G01N 33/6893; C12N 2310/141; C12Q 2600/178; C12Y 203/02; G01N 2333/4704; G01N 2333/7158; G01N 2333/91057; G01N 2800/52

(57) ABSTRACT

Embodiments of the present disclosure provide a method for treating cellular inflammation induced by AFB1, comprising administering to a patient an agent that increases a content or an expression level of miR-9, wherein the agent that increases the content or the expression level of miR-9 is miR-9 mimics or an expression vector containing miR-9, a nucleotide sequence of the miR-9 mimics is shown in SEQ ID No. 5 and a SEQ ID No. 6, and a nucleotide sequence of the miR-9 is shown in SEQ ID No. 5. The present disclosure increases the content or the expression level of miR-9 to antagonize an upstream signaling, block an occurrence of inflammatory response, and inhibit a pro-inflammatory effect of AFB1 on glomerular podocyte.

### CROSS REFERENCE TO RELATED APPLICATIONS

The present disclosure claims priority to Chinese patent application No. 202310730348.5, filed Jun. 20, 2023, and the entirety of which is fully incorporated herein by reference.

### SEQUENCE LISTING

The instant application contains a Sequence Listing which has been submitted electronically in XML format and is hereby incorporated by reference in its entirety. The XML copy, created on Jun. 10, 2024, is named “2024 Jun. 20-Sequece Listing-64001-H008US00” and is 40,785 bytes in size.

### TECHNICAL FIELD

The present disclosure relates to the field of molecular detection technology, and in particular, to a method for treating cellular inflammation induced by AFB1.

### BACKGROUND

MicroRNA (miRNA) is a small non-coding RNA that can post-transcriptionally regulate gene expression related to cell growth, differentiation, and disease mechanisms. Research has found that microRNA-9 (miR-9) targets an inflammatory chemokine receptor CXCR4. By interacting with the C-X-C motif of CXCR4, miR-9 mediates related signaling pathways and can inhibit apoptosis during high glucose-induced injury in human umbilical vein endothelial cells. In contrast, an expression level of miR-9 in glomerular podocytes is decreased in a mouse model of diabetes induced by streptozotocin. These findings suggest a possible correlation between miR-9 and glomerular podocyte injury. Furthermore, it has been reported that CXCR4 can increase an expression level of thioredoxin-interacting protein (TXNIP) and an expression level of NOD-like receptor protein 3 (NLRP3), thereby affecting the secretion of downstream inflammatory factors. Removing CXCR4 can reduce a TXNIP level, indicating that TXNIP is a downstream effector protein in the CXCR4 signaling pathway. In summary, CXCR4 may trigger inflammatory responses by regulating the TXNIP/NLRP3 inflammasome signaling axis.

Aflatoxin B1 (AFB1) is a fungal secondary metabolite and one of the most common environmental carcinogens. It is classified as a Group 1 human carcinogen by the International Agency for Research on Cancer (IARC) and is extremely hazardous. Although AFB1 undergoes metabolism by hepatic microsomal enzymes, the residual portion is excreted by the kidneys, indicating that AFB1 is likely to be a significant cause of inducing renal functional impairment. Therefore, it is urgent to clarify the specific molecular mechanism by which AFB1 induces renal inflammation. Based on previous research findings, whether down-regulation of miR-9 can participate in glomerular podocyte inflammation induced by AFB1 through the CXCR4/TXNIP/NLRP3 inflammasome pathway remains to be elucidated. RelA (also known as p65) is a subunit of NF-κB, a transcription factor derived from the precursor p105 and processed from p52. It forms a heterodimer with the NF-κB subunit p50, known as RelA-p50, which facilitates nuclear translocation and plays crucial roles in metabolic disorders, tumorigenesis, viral infections, and other biological processes. It has been found that RelA is also involved in regulating gene expression of miRNA. However, it remains to be explored whether RelA is involved in regulating the expression level of miR-9, which in turn contributes to glomerular podocyte inflammation induced by AFB1.

Accordingly, it is desired to provide a method for treating cellular inflammation induced by AFB1, targeting and effectively treating glomerular podocyte inflammation induced by AFB1.

### SUMMARY

One or more embodiments of the present disclosure provide a method for treating cellular inflammation induced by AFB1, comprising administering to a patient an agent that increases a content or an expression level of miR-9, wherein the agent that increases the content or the expression level of miR-9 is miR-9 mimics or an expression vector containing miR-9, a nucleotide sequence of the miR-9 mimics is shown in SEQ ID No. 5 and a SEQ ID No. 6, and a nucleotide sequence of the miR-9 is shown in SEQ ID No. 5.

In some embodiments, the increasing the content or the expression level of miR-9 results in at least one of the following to achieve a therapeutic effect:
(1) antagonizing an interaction between RelA induced by AFB1 and E3 ubiquitin ligase TRIM7;
(2) silencing expression of the E3 ubiquitin ligase TRIM7;
(3) antagonizing cellular inflammation induced by a signaling axis of a chemokine receptor CXCR4/TXNIP/NLRP3;
(4) restoring or increasing expression of a transcription factor RelA;
(5) suppressing ubiquitinated degradation of the transcription factor RelA; and
(6) suppressing gene expression of the CXCR4.

In some embodiments, before administering to the patient the agent that increases the content or the expression level of miR-9, an expression level of miR-9 in a biological sample of the patient is detected.

In some embodiments, an expression level of molecules related to signaling axes of RelA, TRIM7, and CXCR4/TXNIP/NLRP3 in the biological sample of the patient is detected after administering to the patient the agent that increases the content or the expression level of miR-9.

In some embodiments, the detecting the expression level of miR-9 includes detecting a gene expression level, an mRNA expression level, or a protein expression level.

In some embodiments, the detecting the expression level of miR-9 in the biological sample of the patient includes performing a polymerase chain reaction (PCR) detection using a forward primer whose nucleotide sequence is shown in SEQ ID No. 3 and a reverse primer whose nucleotide sequence is shown in SEQ ID No. 4.

In some embodiments, the cellular inflammation is glomerular podocyte inflammation.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1A is a diagram illustrating relative expression levels of miR in glomeruli and MPC-5 when treating the glomeruli (n=6) at 0, 2, and 4 weeks using AFB1 of 0.75 mg/kg and treating the MPC-5 (n=6) using AFB1 of 5, 10, and 20 μM, respectively, by adopting Quantitative Reverse Transcription Polymerase Chain Reaction (qRT-PCR) according to embodiments of the present disclosure;

FIG. 1B is a diagram comparing sequences of binding sites between miR-9 and CXCR4 among little mice, big mice, and humans according to embodiments of the present disclosure;

FIG. 1C is a diagram illustrating a potential target map of miR-9 on CXCR4 binding sites and CXCR4 and a schematic diagram illustrating luciferase reporter plasmids of pMIR-CXCR4 binding site and luciferase reporter plasmids of pMIR-CXCR4 mutant binding site.

FIG. 2A is a statistical diagram illustrating a relative expression level of miR-9 after a miR-9 mimics transfected with MPC-5 (n=6) using qRT-PCR according to embodiments of the present disclosure;

FIG. 2B is a statistical diagram illustrating a relative protein expression level of CXCR4 after the miR-9 mimics transfected with MPC-5 (n=6) by adopting Western blot according to embodiments of the present disclosure;

FIG. 2C is a diagram illustrating a relative protein expression level of CXCR4, nephrin, podocin, and NLRP3, ASC, Cleaved, ASC, and Casp1, Casp1, and TXNIP by adopting Western blot according to embodiments of the present disclosure;

FIG. 2D is a statistical diagram illustrating a relative expression level and a protein content of IL-1β, IL-6, and TNF-α mRNA by adopting qRT-PCR and ELISA; where “*” indicates p<0.05 compared to a corresponding cell control group; and “#” indicates p<0.05 compared to AFB1 and a mimic cell control group;

FIG. 3A is a diagram illustrating a relative expression level of miR-9 after CAG-GFP-miR-9 or CAG-GFP luciferase control vector transfected with glomerulus treated with AFB1 of 0.75 mg/kg by adopting qRT-PCR according to embodiments of the present disclosure;

FIG. 3B is a statistical diagram illustrating a relative protein expression level of CXCR4, nephrin, podocin, and NLRP3, ASC, Cleaved-casp1, and TXNIP in glomeruli (n=6) by adopting Western manner according to embodiments of the present disclosure;

FIG. 3C is a statical diagram illustrating detecting a protein content of IL-1β, IL-6 and TNF in glomeruli by adopting ELISA according to embodiments of the present disclosure; where “*” indicates p<0.05 compared with a corresponding normal animal; “#” indicates p<0.05 compared with AFB1 and a rAAv9-CAG-GFP control group;

FIG. 3D is a diagram illustrating HE staining for inflammatory cell infiltration in glomeruli treated with AFB1 for 4 weeks (n=3, scale bar is 20 μm, arrows indicate inflammatory cytokine infiltration and thickening of a thylakoid membrane in glomeruli) according to embodiments of the present disclosure;

FIG. 3E is a diagram illustrating proportions of neutrophils expressing IFN-γ and Gr-1 and proportions of macrophages expressing CD68 and CD86 in glomeruli (n=5) of each experimental group at week 4 detected by flow cytometry according to the embodiment of the present disclosure;

FIG. 4A is a statistical diagram illustrating a relative protein expression level of RelA in glomeruli and MPC-5 when treating glomeruli (n=6) at 0, 2, and 4 weeks using AFB1 of 0.75 mg/kg and treating MPC-5 (n=6) using AFB1 of 5, 10, and 20 μM, respectively, by adopting Western blot according to embodiments of the present disclosure;

FIG. 4B is a statistical diagram illustrating a relative expression level of RelA-regulated miRNA (n=6) in MPC-5 transfected with RelA siRNA or in MPC-5 in a control agent and a relative expression level of miR-9 in MPC-5 (n=6) transfected with LV5-RelA by adopting qRT-PCR according to embodiments of the present disclosure;

FIG. 4C is a diagram illustrating a binding situation between a miR-9 prompter and RelA in MPC-5 under conditions exposed or unexposed to AFB1 and with or without MG-132 by adopting ChIP-PCR according to embodiments of the present disclosure; where “*” indicates p<0.05 compared to a corresponding cell control group; “#” indicates p<0.05 compared to AFB1 and a LV5-GFP cell group;

FIG. 4D is a heatmap illustrating proteins that interact with RelA detected by IP-MS according to embodiments of the present disclosure;

FIG. 4E is an electrophoresis diagram illustrating an interaction between RelA, TRIM7, and ubiquitin detected by immunoprecipitation (IP) combined with Western blot according to embodiments of the present disclosure; and

FIG. 4F is an electrophoresis diagram illustrating RelA and a ubiquitination level of RelA in MPC-5 transfected with TRIM7 siRNA detected by immunoprecipitation (IP) combined with Western blot according to embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system”, “device”, “unit” and/or “module” as used herein is a way to distinguish between different components, elements, parts, sections, or assemblies at different levels. However, the words may be replaced by other expressions if other words accomplish the same purpose.

As shown in the present disclosure and in the claims, unless the context clearly suggests an exception, the words “a”, “one”, “one kind”, and/or “the” do not refer specifically to the singular, but may also include the plural. Generally, the terms “including” and “comprising” suggest only the inclusion of clearly identified steps and elements that do not constitute an exclusive list, and the method or apparatus may also include other steps or elements.

AFB1 is a secondary metabolite of Aspergillus flavus and Aspergillus parasiticus, which is extremely toxic and can severely damage the body's immune system and trigger cellular inflammation. Studies have shown that AFB1 induces glomerular podocyte inflammation during renal excretion, but the mechanism of induction remains to be elucidated. miR-9, a small non-coding RNA that plays a role in the regulation of gene transcription, may be relevant to glomerular podocyte injury. Clarifying the role of miR-9 in the inflammatory pathway could help develop therapeutic drugs related to glomerular podocyte inflammation induced by AFB1.

Embodiments of the present disclosure provide a method for treating cellular inflammation induced by AFB1, comprising administering to a patient an agent that increases a content or an expression level of miR-9, wherein the agent that increases the content or the expression level of miR-9 is miR-9 mimics or an expression vector containing miR-9, a nucleotide sequence of the miR-9 mimics is shown in SEQ ID No. 5 and a SEQ ID No. 6, and a nucleotide sequence of the miR-9 is shown in SEQ ID No. 5.

As used in the present disclosure, the miR-9 is mmu-miR-9-5p MIMAT0000142. miR-9 mimics are chemically synthesized mimics of the miR-9 that may simulate activity of miR-9 in cells to study the gain of function (GOF) effect. In some embodiments, administering the miR-9 mimics to the patient may increase the content or the expression level of miR-9 in the patient.

In some embodiments, the cellular inflammation induced by AFB1 may include glomerular podocyte inflammation.

In some embodiments, increasing the content or the expression level of the miR-9 results in at least one of the following to achieve a therapeutic effect:
(1) antagonizing an interaction between RelA induced by AFB1 and E3 ubiquitin ligase TRIM7;
(2) silencing expression of the E3 ubiquitin ligase TRIM7;
(3) antagonizing cellular inflammation induced by a signaling axis of a chemokine receptor CXCR4/TXNIP/NLRP3;
(4) restoring or increasing expression of a transcription factor RelA;
(5) suppressing ubiquitinated degradation of the transcription factor RelA; and
(6) suppressing gene expression of the CXCR4.

CXCR4 is an inflammatory chemokine receptor. In some embodiments, CXCR4 gene is a target of the miR-9. In some embodiments, miR-9 may targeted bind to a C-X-C motif of the CXCR4 gene. TXNIP refers to thioredoxin-interacting protein. NLRP3 refers to NOD-like receptor heat protein structural domain-associated protein 3. RelA is a member of the NF-κB family of eukaryotic transcription factors, which plays an important role in the regulation of inflammation, tumor, metabolism, and immune response, among other important life activities and the development of related diseases.

In some embodiments, the expression level of miR-9 in a biological sample of the patient is detected before administering to the patient the agent that increases the content or the expression level of miR-9.

In some embodiments, detecting the expression of miR-9 in the biological sample of the patient includes detecting a gene expression level, an mRNA expression level, or a protein expression level.

In some embodiments, a manner to detect the expression level of miR-9 in the biological sample of the patient includes performing a polymerase chain reaction (PCR) detection using a forward primer whose nucleotide sequence is shown in SEQ ID No. 3 and a reverse primer whose nucleotide sequence is shown in SEQ ID No. 4.

In some embodiments, an expression level of molecules related to signaling axes of RelA, TRIM7, and CXCR4/TXNIP/NLRP3 in the biological sample of the patient is detected after administering to the patient the agent that increases a content or an expression level of miR-9.

The present disclosure does not specifically limit a detection manner, and the detection manner may include, but is not limited to, qRT-PCR, Western blot, or ELISA.

Preferably, the primer described in the embodiments of the present disclosure also includes a stem-loop sequence for reverse transcription, a nucleotide sequence of the stem-loop sequence is as shown in SEQ ID No. 45: CCTGTTGTCTCCAGCCACAAAAGAGCACAATATTTCAGGAGACAACAGG.

Beneficial effects that may result from embodiments of the present disclosure include, but are not limited to that: (1) a content or an expression level of miR-9 in a patient is increased by administering miR-9 mimics or an expression vector containing miR-9, so that miR-9 can inhibit expression of its target gene chemokine receptor CXCR4 by that regulates the TXNIP/NLRP3 signaling axis to induce glomerular podocyte inflammation; (2) AFB1 can promote the interaction of RelA and E3 ubiquitin ligase TRIM7 to increase the ubiquitination degradation of the transcription factor RelA and down-regulate the expression level of miR-9, which modulates its inhibitory effect on CXCR4. The embodiments of the present disclosure, by increasing the expression level of miR-9, can antagonize the upstream signaling, block the occurrence of inflammatory response, and inhibit the pro-inflammatory effect of AFB1 on glomerular podocyte.

In order to further illustrate the present disclosure, the application of miR-9 and a miR-9 mimic provided herein in the preparation of medicines for diagnosing and/or treating cellular inflammation is described in detail below in conjunction with the embodiments, but they are not construed as limiting the scope of protection of the present disclosure.

### EMBODIMENTS

### Embodiment 1: AFB1 Upregulates CXCR4 by Inhibiting miR-9 in Glomeruli and Glomerular Podocyte (MPC-5)

(1) glomeruli (n=6) were treated with AFB1 of 0.75 mg/kg at 0, 2, and 4 weeks;
(2) MPC-5 (n=6) was treated with AFB1 of 5, 10, and 20μ M;
(3) Relative expression levels of miR-9 in the glomeruli treated with (1) and in the MPC-5 treated with (2) were detected by adopting a qRT-PCR manner, respectively. u6 RNA was used as an internal reference for miRNA, a sequence of a forward primer of U6 gene was shown in SEQ ID NO.1 in Table 1, and a sequence of a reverse primer of U6 gene was shown in SEQ ID NO.2. A sequence of a forward primer of miR-9 was shown in SEQ ID NO.3, a sequence of a reverse primer of miR-9 was shown in SEQ ID NO.4, and a sequence of a primer of a stem-loop sequence that is used to increase a length of a miR-9 reverse transcription product was shown in SEQ ID No.45.

TABLE 1
Sequences of primers for detecting a relative
expression level of miR-9

| genetics | Sequences of primers (5′ → 3′) | SEQ ID No. |
|---|---|---|
| U6 | F: GCTTCGGCAGCACATATACTAAAAT | 1 |
| | R: CGCTTCACGAATTTGCGTGTCAT | 2 |
| miR-9 | F: CGGGCTCTTTGGTTATCTAGC | 3 |
| | R: CAGCCACAAAAGAGCACAAT | 4 |
| stem-loop | CCTGTTGTCTCCAGCCACAAAAGAGCA | 45 |
| sequence | CAATATTTCAGGAGACAACAGG | |

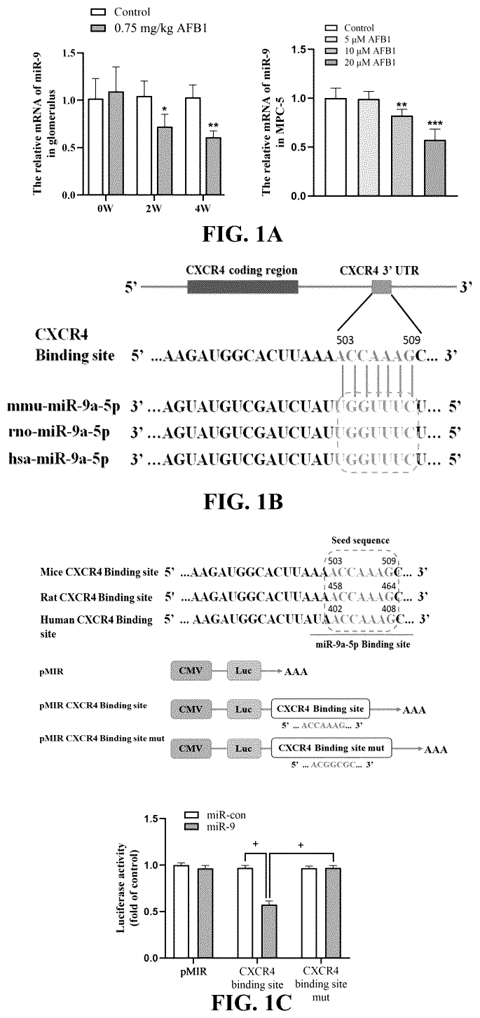

The results are shown in FIG. 1. The expression levels of miR-9 both in the glomeruli and cells significantly reduced after treatment with AFB1 (FIG. 1A). Prediction of miR-9 target gene using TargetScan 5.2 (http://www.targetscan.org) revealed that CXCR4 is one of the target genes of miR-9, and miR-9 functions by binding to 3′-UTR of CXCR4 gene (FIG. 1B).

To validate the binding of miR-9 to the CXCR4 gene, 3′-UTR targeting sequences of a wild-type CXCR4 gene and a mutant-type CXCR4 gene (target site ACCAAAG was mutated to ACGGCGC) were constructed into a luciferase reporter vector pMIR to obtain recombinant luciferase reporter vectors of the wild-type and the mutant-type. Subsequently, miR-9 and negative control miRNAs (a sequence of a primer as shown in SEQ ID NO.7 and SEQ ID NO.8) were co-transfected with the recombinant luciferase reporter vector of the wild-type or the mutant type into MPC-5 cells, and results were as shown in FIG. 1C. The targeted binding of miR-9 to the 3′-UTR of the wild-type CXCR4 led to a relative decrease in fluorescence values; on the contrary, miR-9 was unable to bind to the 3′-UTR of the mutant-type CXCR4 and the fluorescence values were unchanged, suggesting that miR-9 can inhibit gene expression of CXCR4 through a 3′-UTR binding site. The control miRNAs were unable to targetedly bind to either the 3′-UTR of the wild-type CXCR4 or the 3′-UTR of the mutant-type CXCR4, and the fluorescence values were changed.

### Embodiment 2: AFB1 Upregulates Expression of CXCR4 Through Downregulating Expression of miR-9, which in Turn Activates the TXNIP/NLRP3 Pathway to Induce MPC-5 Inflammatory Response

Related agents include β-actin antibody (Abcam), nephrin antibody (Abcam), podocin antibody (Abcam), CXCR4 antibody (Abcam), ASC antibody (Abcam), NLRP3 antibody (Abcam), Pro-caspase1 antibody (CST), Cleaved-caspase 1 antibody (CST), TXNIP antibody (Abcam), goat anti-rabbit IgG H&L (HRP) (CST), and ELISA kits (R&D Systems Inc.).

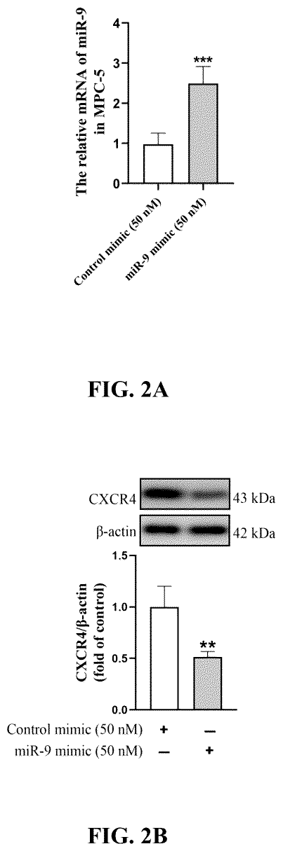

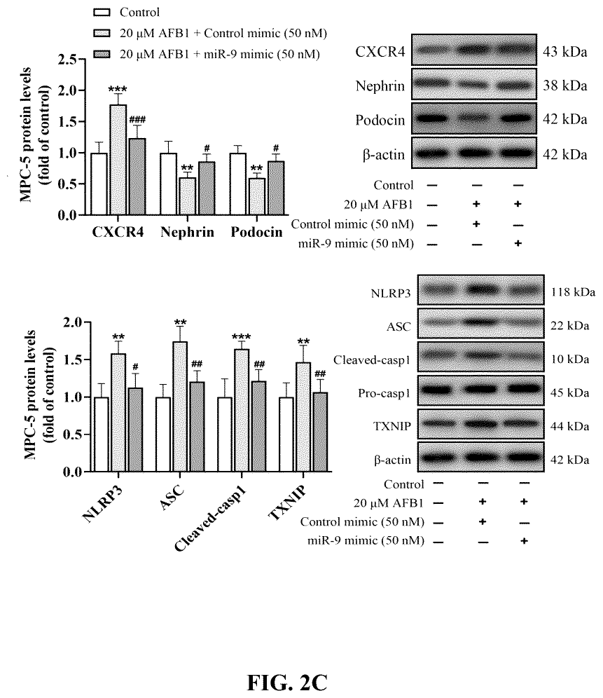

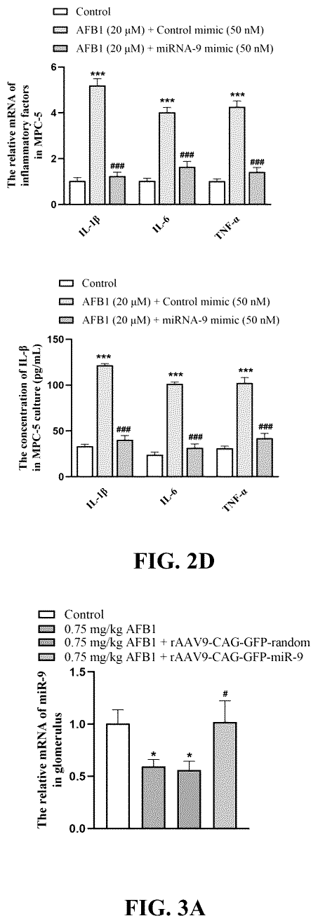

To verify the role of miR-9 in regulating CXCR4 in AFB1-treated MPC-5 cells, a miR-9 mimic was transiently transfected into MPC-5 cells for 24 h. The transfection efficiency of the miR-9 mimic was detected by the qRT-PCR manner. A sequence of a primer of miR-9 mimic is shown in Table 2, including a sequence of a forward primer as shown in SEQ ID NO.5 and a sequence of a reverse stream as shown in SEQ ID NO.6. Results shown in FIG. 2 showed a significant increase in an expression level of miR-9 (FIG. 2A) and a significant decrease in a relative protein expression level of CXCR4 (FIG. 2B).

Compared to MPC-5 cells co-treated with a miR-9 control mimic and AFB1, MPC-5 cells co-treated with miR-9 mimic and AFB1 (24 h) showed a significant increase in a relative protein expression level of nephrin and podocin and a significant decrease in CXCR4, TXNIP, NLRP3, ASC, and Cleaved-casp1 (FIG. 2C). miR-9 mimic transfection led to a reduction in a relative expression level and a protein content of IL-1β, IL-6, and TNF-α mRNA in AFB1-treated renal peduncle cells (FIG. 2D), where a sequence of a primer for detecting the relative expression level of IL-1β, IL-6, and TNF-α mRNA is shown in Table 2. Results further demonstrated the negative regulatory role of miR-9 in glomerular podocytes inflammation induced by AFB1.

TABLE 2
Sequence listing

| genetics | Sequence (5′ → 3′) | SEQ ID No. |
|---|---|---|
| miRNA-9 | F: UCUUUGGUUAUCUAGCUGUAUGA | 5 |
| mimic | R: AUACAGCUAGAUAACCAAAGAUU | 6 |
| Negative | F: GCCGAGTCTTTGGTTATCTAGC | 7 |
| control | R: AGTGCAGGGTCCGAGGTATT | 8 |
| IL-1β | F: GCCACCTTTTGACAGTGATGAG | 9 |
| | R: AAGGTCCACGGGAAAGACAC | 10 |
| IL-6 | F: GTCCTTCCTACCCCAATTTCCA | 11 |
| | R: TAACGCACTAGGTTTGCCGA | 12 |
| TNF-α | F: CATCTTCTCAAAATTCGAGTGACAA | 13 |
| | R: TGGGAGTAGACAAGGTACAACCC | 14 |
| U6 | F: GCTTCGGCAGCACATATACTAAAAT | 1 |
| | R: CGCTTCACGAATTTGCGTGTCAT | 2 |
| β-actin | F: AGAGGGAAATCGTGCGTGAC | 15 |
| | R: CAATAGTGATGACCTGGCCGT | 16 |

### Embodiment 3: AFB1 Induces Glomerular Podocyte Inflammation by Down-Regulating miR-9 and Activating TXNIP/NLRP3 Pathway by Increasing CXCR4

Relevant agents include fluorescently labeled Gr-1 antibody (CST), fluorescently labeled IFN-γ antibody (CST) or CD86 antibody (fluorescent secondary antibody: goat anti-rabbit IgG) (Abcam) and CD68 antibody (fluorescently secondary antibody: goat anti-rabbit IgG) (Abcam), and the rest of the same agents as above.

To understand the ability of miR-9 in down-regulating the CXCR4/TXNIP/NLRP3 inflammatory vesicle pathway during podocytes inflammation induced by AFB1, a 4-week-old mouse was treated with AFB1 of 0.75 mg/kg, and rAAV9-CAG-GFP-miR-9 or CAG-GFP luciferase control vector was transfected into the treated mouse, and an expression level of miR-9 in glomeruli after transfection was detected by qRT-PCR.

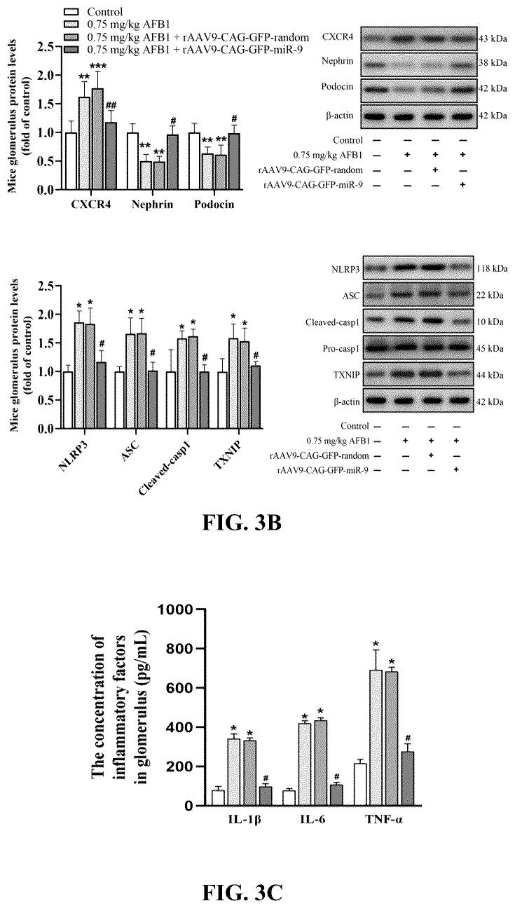

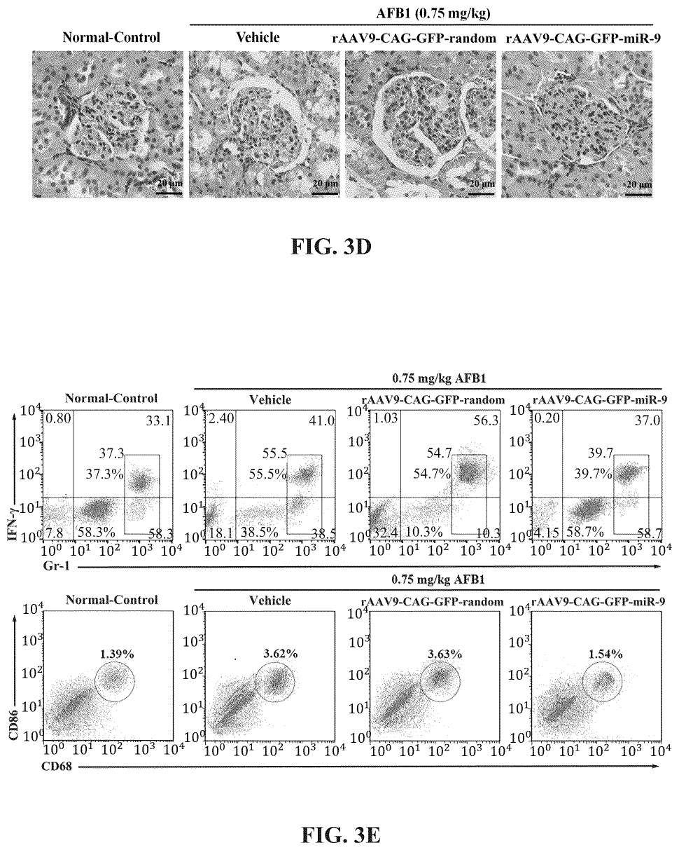

As shown in FIG. 3, the expression level of miR-9 significantly increased after transfection of AFB1-treated glomeruli using rAAV9-CAG-GFP-miR-9 (FIG. 3A). Expression of miR-9 resulted in a significant reduction in a protein expression level of CXCR4 and a significant increase of nephrin and podocin, as well as suppressed the increase of TXNIP, NLRP3, ASC, and Cleaved-casp1 in the AFB1-treated glomeruli (FIG. 3C).

In addition, increased miR-9 also attenuated glomerular inflammation induced by AFB1, as evidenced by decreased secretion of inflammatory cytokines IL-1β, IL-6, and TNF-α (FIG. 3D) and attenuated inflammatory cell infiltration (FIG. 3E and FIG. 3F).

### Embodiment 4 AFB1 Reduces a Content of RelA and Downregulates Expression of miR-9 by Promoting Hydrolysis of RelA Ubiquitin-Dependent Protein

Relevant agents include RelA antibody (Abcam), TRIM7 antibody (Abcam), Lamin A antibody (Abcam), Ubiquitin antibody (Abcam), and the rest of the same agents as above.

To investigate whether RelA is involved in the reduction of the expression level of miR-9 in glomerular podocytes by AFB1, relative protein expression levels of RelA in glomeruli at 0, 2, and 4 weeks treated with AFB1 of 0.75 mg/kg, and MPC-5 cells treated with AFB1 of 5, 10, 20 uM were detected, respectively, by adopting a Western blot manner.

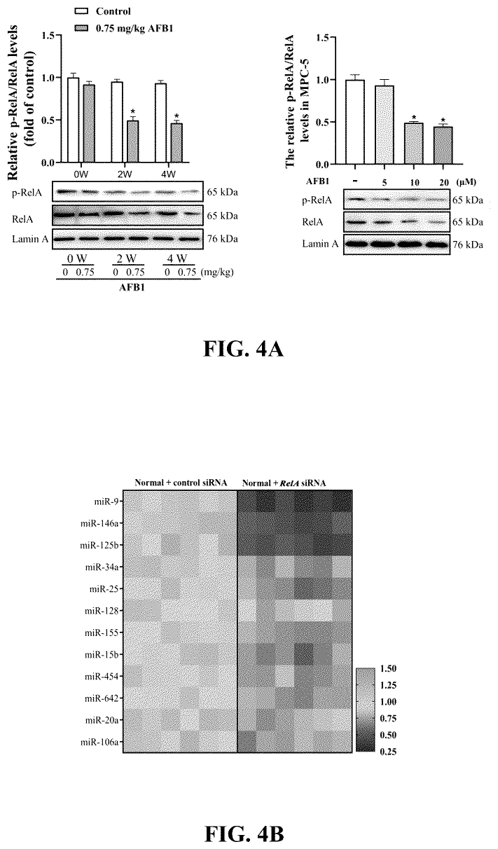

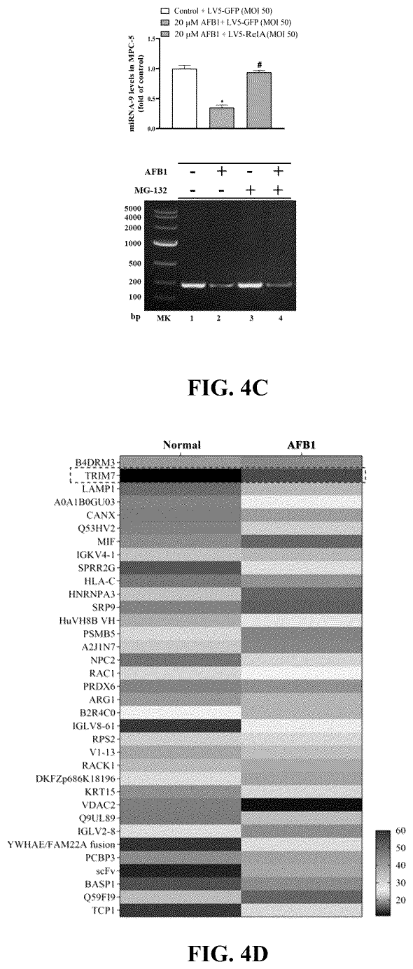

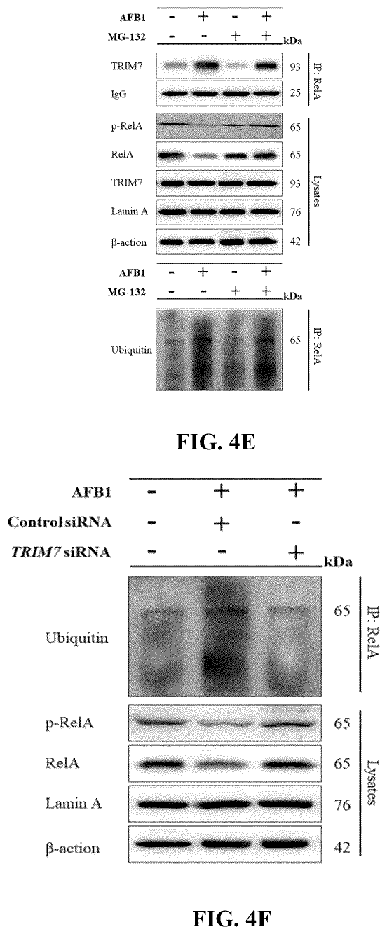

Results in FIG. 4 show a significant reduction of p-RelA/RelA in glomeruli and MPC-5 cells after treated with AFB1 (FIG. 4A).

To investigate a relationship between RelA and the expression level of miR-9 in MPC-5 cells, AFB1-treated MPC-5 cells were transfected with RelA siRNA or a control agent, and a relative expression level of RelA-regulated miRNA in the transfected MPC-5 cells was detected by a qRT-PCR manner; LV5-GFP or LV5-RelA were used to transfect MPC-5 cells treated with AFB1 of 20 uM, and the relative expression level of miR-9 in transfected MPC-5 cells was detected by the qRT-PCR manner. Sequences of primers for detecting a relative expression level of miRNA are shown in Table 3. Results showed that overexpression of RelA can counteract the inhibitory effect of AFB1 on miR-9 (FIG. 4B), suggesting that AFB1 may reduce the expression level of miR-9 by down-regulating RelA and thereby reducing miR-9.

In order to analyze the binding of RelA and a miR-9 promoter region, a ChIP-PCR manner was used to detect the binding of a miR-9 promoter to RelA in MPC-5 cells under conditions exposed or unexposed to AFB1 and with or without MG-132, i.e., the MPC-5 cells were fixed with 1% formaldehyde for 10 min, cells were collected, nuclei were sonicated and incubated overnight at 4° C. with RelA antibody (CST) or control IgG, immunoprecipitation was performed, DNA was eluted from the precipitated immune complexes, and DNA fragment of the miR-9 promoter bound to RelA was amplified by PCR, then relative quantification was performed using nucleic acid gel electrophoresis. Results showed that AFB1 reduced RelA binding in the miR-9 promoter region in the MPC-5 cells with or without the addition of a proteasome inhibitor MG-132 (FIG. 4C), suggesting that the reduced RelA plays a role in miR-9 downregulation induced by miR-9.

For the analysis of RelA-binding proteins, IP-MS was used to detect proteins interacting with RelA. After incubating THE MPC-5 cells with RelA antibody overnight at 4° C., MPC-5 lysates were mixed with Protein A/G agarose beads (Amersham Biosciences) and then sodium dodecyl sulfate-polyacrylamide gel electrophoresis (SDS-PAGE) analysis and Caumas blue staining were performed for isolation of target proteins. Then, protein samples were sent to Suzhou Mass-elife Biotechnology Co. Ltd (Suzhou, China) for further analysis. For liquid chromatography-mass spectrometry (LC-MS) analysis, proteins were re-solubilized in a mixture of acetonitrile and water, and sample data was analyzed using an ultra-high-performance liquid chromatography (UHPLC) system coupled to a high-resolution mass spectrometer in Mass-elife Biotech Co. Ltd. of Suzhou (Suzhou, China). Identified proteins were analyzed for abundance using Byonic 2.13.17 (Protein Metrics, California, USA) to retrieve MS raw files on the uniport human protein database. Results showed that TRIM7 in an AFB1-treated group was significantly enriched in AFB1-treated MPC-5 cells compared with a corresponding control group (FIG. 4D).

Subsequently, the interaction of RelA with TRIM7, ubiquitin, in the MPC-5 cells under conditions exposed or unexposed to AFB1 and with or without MG-132 was revealed by Co-IP. Namely, MPC-5 whole cell lysates were obtained by ultracentrifugation at 12,000×g for 20 min at 4° C., then incubated overnight at 4° C. with RelA antibody and protein A/G agarose beads (Amersham Biosciences). Afterwards, immune complexes were washed with lysis buffer, eluted with sodium sample buffer, and analyzed by WB. Results showed that AFB1 promoted the binding of RelA and TRIM7, which increased the ubiquitination of RelA and down-regulated an expression level of RelA protein (FIG. 4E). Finally, TRIM7 siRNA was transfected into AFB1-exposed MPC-5 cells, and RelA and a ubiquitination level of ReIA in the transfected MPC-5 cells were detected by immunoprecipitation combined with Western blot. Results showed that silencing TRIM7 reduced the ubiquitination level of RelA in the MPC-5 cells but restored a protein level of RelA after being treated with AFB1 (FIG. 4F).

In summary, AFB1 treatment led to the down-regulation of the expression level of miR-9 by enhancing RelA binding to TRIM7 and thereby increasing degradation of ubiquitin-dependent RelA.

TABLE 3
Sequence listing

| genetics | Sequences of primers (5′ → 3′) | SEQ ID No. |
|---|---|---|
| RelA | F: CGTGAAGAAGAAGGCCCGCAAGT | 17 |
| | R: CTTGGAGGCGGCAACAGCTTTAGTA | 18 |
| U6 | F: GCTTCGGCAGCACATATACTAAAAT | 1 |
| | R: CGCTTCACGAATTTGCGTGTCAT | 2 |
| miR- | F: CGGGCTGAGAACTGAATTCC | 19 |
| 146a | R: CAGCCACAAAAGAGCACAAT | 20 |
| miR- | F: CGGGCTCCCTGAGACCCTAA | 21 |
| 125b | R: CAGCCACAAAAGAGCACAAT | 22 |
| miR- | F: CGGGCTGGCAGTGTCTTAGC | 23 |
| 34a | R: CAGCCACAAAAGAGCACAAT | 24 |
| miR- | F: CGGGCAGGCGGAGACTTGGG | 25 |
| 25 | R: CAGCCACAAAAGAGCACAAT | 26 |
| miR- | F: CGGGCCGGGGCCGTAGCAC | 27 |
| 128 | R: CAGCCACAAAAGAGCACAAT | 28 |
| miR- | F: CGGGCTTAATGCTAATTGTGA | 29 |
| 155 | R: CAGCCACAAAAGAGCACAAT | 30 |
| miR- | F: CGGGCTAGCAGCACATCATG | 31 |
| 15b | R: CAGCCACAAAAGAGCACAAT | 32 |
| miR- | F: CGGGCTGGCAGTGTCTTAGC | 33 |
| 454 | R: CAGCCACAAAAGAGCACAAT | 34 |
| miR- | F: CGGGCTAGCAGCACATCATG | 35 |
| 642 | R: CAGCCACAAAAGAGCACAAT | 36 |
| miR- | F: CGGGCTAAAGTGCTTATAGTG | 37 |
| 20a | R: CAGCCACAAAAGAGCACAAT | 38 |
| miR- | F: CGGGCCAAAGTGCTAACAGTG | 39 |
| 106a | R: CAGCCACAAAAGAGCACAAT | 40 |
| RelA | F: GCAGUUUGAUGCUGAUGAATT | 41 |
| siRNA | R: UUCAUCAGCAUCAAACUGCTT | 42 |
| TRIM7 | F: GAUUGCUGAAGAAGUUCAATT | 43 |
| siRNA | R: UUGAACUUCUUCAGCAAUCTT | 44 |

Note:
The RNA sequences shown in SEQ ID NO. 41, SEQ ID NO. 42, SEQ ID NO. 43, and SEQ ID NO. 44 are artificially synthesized, and the “TT” at the end of the sequences represents thymidine which has been artificially added. Therefore, in the sequence listing submitted along with the present disclosure, the “TT” at the end of the RNA sequences shown in SEQ ID NO. 41, SEQ ID NO. 42, SEQ ID NO. 43, and SEQ ID NO. 44 represents thymidine.

AFB1 increases the ubiquitination degradation of a transcription factor RelA by promoting the interaction of RelA and the E3 ubiquitin ligase TRIM7 (tripartite motif containing 7), which subsequently down-regulates the expression level of miR-9; miR-9 induces glomerular podocyte inflammation through its action on signaling axis of chemokine receptor CXCR4/TXNIP/NLRP3.

### Embodiment 5: Detection of Expression of miR-9 and its Related Molecules and Diagnosis of Glomerular Podocyte Inflammation Induced by AFB1

An application of RT-PCR to detect an expression level of miR-9, and an application of RT-PCR or Western blot to detect expression levels of molecules related to signaling axes of RelA and CXCR4/TXNIP/NLRP3 may diagnose kidney injury induced by AFB1.

By controlling with normal tissue cells, the down-regulation of the expression level of miR-9 and low expression of RelA associated with miR-9 and the high expression of molecules related to the signaling axes of CXCR4/TXNIP/NLRP3 indicated that AFB1 induced glomerular podocyte to generate an inflammatory response.

### Embodiment 6: Molecules or Strategies to Inhibit Glomerular Podocyte Inflammation Induced by AFB1 by Targeting a Signaling Pathway of RelA-miR-9-CXCR4/TXNIP/NLRP3

By designing and synthesizing miR-9 mimics, or expressing miR-9 via a vector, and applying viral or non-viral technologies to mediate their transfection into in vitro or in vivo models, pro-inflammatory effect of AFB1 on glomerular podocyte was suppressed; and by silencing expression of E3 ubiquitin ligase TRIM7, an inflammatory signaling pathway was more effectively affected, etc.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various parts described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by +20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. A method for treating cellular inflammation induced by AFB1, comprising:
administering to a patient an agent that increases a content or an expression level of miR-9, wherein the agent that increases the content or the expression level of miR-9 is miR-9 mimics or an expression vector containing miR-9, a nucleotide sequence of the miR-9 mimics is shown in SEQ ID No. 5 and a SEQ ID No. 6, and a nucleotide sequence of the miR-9 is shown in SEQ ID No. 5.

2. The method of claim 1, wherein increasing the content or the expression level of miR-9 results in at least one of the following to achieve a therapeutic effect:
(1) antagonizing an interaction between RelA induced by AFB1 and E3 ubiquitin ligase TRIM7;
(2) silencing expression of the E3 ubiquitin ligase TRIM7;
(3) antagonizing cellular inflammation induced by a signaling axis of a chemokine receptor CXCR4/TXNIP/NLRP3;
(4) restoring or increasing expression of a transcription factor RelA;
(5) suppressing ubiquitinated degradation of the transcription factor RelA; and
(6) suppressing gene expression of the CXCR4.

3. The method of claim 1, further comprising detecting, before administering to the patient the agent that increases the content or the expression level of miR-9, an expression level of miR-9 in a biological sample of the patient.

4. The method of claim 3, further comprising detecting an expression level of molecules related to signaling axes of RelA, TRIM7, and CXCR4/TXNIP/NLRP3 in the biological sample of the patient after administering to the patient the agent that increases the content or the expression level of miR-9.

5. The method of claim 3, wherein detecting the expression level of miR-9 includes detecting a gene expression level, an mRNA expression level, or a protein expression level.

6. The method of claim 3, wherein the detecting the expression level of miR-9 in the biological sample of the patient includes performing a polymerase chain reaction (PCR) detection using a forward primer whose nucleotide sequence is shown in SEQ ID No. 3 and a reverse primer whose nucleotide sequence is shown in SEQ ID No. 4.

7. The method of claim 1, wherein the cellular inflammation is glomerular podocyte inflammation.
